(19) United States
(12) Patent Application Publication
(10) Pub. No.: US 2024/0350220 A1
(43) Pub. Date: Oct. 24, 2024

(54) MEDICAL COLLABORATIVE ROBOT HAVING ADJUSTABLE ROBOTIC ARM, AND CONTROL METHOD

(71) Applicant: B. Braun New Ventures GmbH, Freiburg im Breisgau (DE)

(72) Inventors: Tim Beyl, Baden-Baden (DE); Shu Chuan Yao, Stuttgart (DE)

(21) Appl. No.: 18/684,758

(22) PCT Filed: Aug. 18, 2022
(86) PCT No.: PCT/EP2022/073104

§ 371 Date: Feb. 19, 2024

(30) Foreign Application Priority Data
Aug. 20, 2021 (DE) 10 2021 121 673.7

(51) Int. Cl. A61B 34/00; A61B 34/37; A61B 90/00

(52) U.S. Cl. CPC A61B 34/77; A61B 34/37; A61B 2090/065

(57) ABSTRACT

A medical collaborative robot, control method, and computer-readable storage medium can be used for actuating a medical end effector during a surgical operation. The robot includes a robot base as a local connection point for the robot; a moving robotic arm connected to the robot base that has more degrees of freedom in the axis space than degrees of freedom for manipulating; an end effector connected to the robotic arm, in particular to a terminal side of the robotic arm; and a control unit adapted to control and actuate at least the robotic arm. At least one input device and/or detector is arranged on the robotic arm. The control unit, when an object is input or detected, is adapted to control the robotic arm via the control unit in such a way that when the robotic arm moves, the end effector maintains its position, in particular its pose.

### CROSS REFERENCE TO RELATED APPLICATIONS

This application is the United States national stage entry of International Application No. PCT/EP2022/073104, filed on Aug. 18, 2022, and claims priority to German Application No. 10 2021 121 673.7, filed on Aug. 20, 2021. The contents of International Application No. PCT/EP2022/073104 and German Application No. 10 2021 121 673.7 are incorporated by reference herein in their entireties.

### FIELD

The present disclosure relates to a medical collaborative robot (cobot) for actuating a medical end effector/manipulator in the field of medical technology, in particular during a surgical procedure on a patient. The collaborative robot has a robot base as a local connection point of the robot, as well as a movable and thus adjustable robotic arm connected to the robot base, which in particular has more degrees of freedom in the axis space than degrees of freedom to be manipulated. An end effector can be attached/coupled to the robotic arm, in particular to a terminal side/to a free end of the robotic arm, in particular articulated or mounted by means of a joint. As the end effector a camera system, in particular an optical camera, can also serve, which takes an image. Furthermore, the robot has a control unit, which is adapted to control at least the robotic arm and to activate it accordingly and to move it accordingly within geometric framework conditions. In addition, the present disclosure relates to a control method and a computer-readable storage medium.

### BACKGROUND

In the field of medicine and medical technology, automation with the associated integration of digitally controllable technical devices is becoming increasingly important. Robots are being used increasingly in surgical procedures to support precise minimally invasive surgery, for example. Here, the robot is not only intended as a stand-alone robot that only performs the operation, but the robot is increasingly being used as a collaborative robot (cobot), i.e. as an assisting or supporting robot, directly in the surgical field, which interacts with a medical professional, in particular the surgeon. For example, the collaborative robot can perform surgical procedures in sections and hands over another surgical procedure to a surgeon as required.

However, this type of interaction between humans and robots poses problems that still need to be solved. Since a robotic arm with an end effector is located in the field of surgery, but a surgeon must also operate in this field, there are inevitably geometric obstructions caused by the robotic arm. For example, the robotic arm may be located in a surgeon's field of vision or access area.

A more recent approach in the prior art is to incorporate a torque sensor in each joint of a robot, which makes it possible to measure a torque per joint and drive. In such robots with torque sensors, a control mode is provided in which the surgeon or user can manually exert a force on a member/segment of the robotic arm, whereby the resulting torque is measured in the joints. The torque is then analyzed using a control algorithm and converted into a resulting movement of the robotic arm. Using information about the kinematics and dynamics of the robot as part of a mathematical model, the interaction force on the robot joints can be calculated in most scenarios. However, this also has the disadvantage that unintentionally applied forces on a robotic arm segment along the entire robotic arm, for example by bumping into it, can also lead to unintentional movements of the robot, as the entire robotic arm transmits the forces to the joints and thus to the torque sensors. Moreover, the movement of the robotic arm only can be controlled inadequately. In addition, the applied force can itself cause a change in the end effector pose (e.g. by deforming the structure of the robot).

### SUMMARY

It is therefore the task of the present disclosure to avoid or at least reduce the disadvantages of the prior art and in particular to provide a medical collaborative robot (cobot), a control method and a computer-readable storage medium which allows interaction with a user in a special simple, intuitive, but also safe manner. A sub-task can be seen in the simple, safe and fast release of a work area by the collaborative robot when required in order to improve interaction between the user and the collaborative robot, or to make access to the surgical field better accessible.

The tasks are solved with respect to a medical collaborative robot according to the invention, with respect to a generic control method according to the invention, and with respect to a computer-readable storage medium.

A basic idea thus consists in providing, in a (collaborative) robot having an articulated robotic arm and end effector, an operating element (an input and/or detection means or operating and/or recognition means, respectively) at such a point on the robotic arm that can be moved easily, but with the kinematics of the robotic arm being controlled by the control unit in such a way that the end effector does not change its position and in particular pose (i.e. its position and orientation/alignment) in absolute space. While the end effector thus remains static exactly in its position respectively pose and, for example, an attached instrument is not moved relative to a patient (at least not a tip), the robotic arm itself can be controlled in a particularly simple and safe manner by way of the input and/or detection means and its pose in space can be changed in order to ensure good access to the surgical field, in particular to a surgeon.

In other words, on the robotic arm, in particular on at least one suitable and specific position/point or area, preferably on a joint and/or bearing of the robotic arm as a connection, at least one input and/or detection means (operating and/or detection means) is arranged/provided and the control unit is specially adapted to control and move the robotic arm in such a way via the control unit when an (operating) input is made by the input means respectively an object is detected by the detection means that the end effector maintains its position, in particular pose, when the robotic arm is moved.

In still other words, a particular requirement of a system respectively robot having an arm/robotic arm is that this robot respectively robotic arm has more degrees of freedom in the axis space than the degrees of freedom to be manipulated, in particular that it is overdetermined. The pose of the robotic arm thus is not determined bijectively by the position or pose (i.e. position and orientation) of the effector. This remaining degree of freedom is taken advantage of in the present case and, in particular, special points are defined on the robotic arm at which the robotic arm can be moved without the effector changing its position. Hence, the segmented robotic arm with a large number of robotic arm segments can be moved by input and/or detection means attached directly to the segments of the robotic arm in such a way that the working range respectively area of intervention remains free for the user, but the end effector retains its position, in particular its pose.

The present disclosure also describes in particular a targeted arrangement/placement of interaction units (input and/or detection means), in particular haptic interaction units, on the robotic arm of the robot, i.e. the structure of the robot, in order to be able to interact particularly advantageously with the collaborative robot.

In still quite different words, a robot, a control unit (with correspondingly stored control algorithms) and at least one input and/or detection means, in particular a pair, are provided, wherein the at least one input and/or detection means is attached, in particular integrated, to one or more members of the robotic arm. In particular, the robot or robotic arm has more degrees of freedom than the task space in which it operates (for example, a robot with seven degrees of freedom (7 DoF) operating in six degrees of freedom (6DoF)). Here, the solution to an inverse kinematic problem usually comprises more than one solution, usually an infinite number of solutions. Typically, one of the solutions is selected on the basis of one criterion or more criteria (in particular, greatest distance to joint boundaries and/or slightest movement of one or more joints). The input and/or detection means is used as an interaction means for the user to optimize the kinematic solution based on a user's current needs. In particular, if parts of the robotic arms restrict the user's working range, the input and/or detection means are used to move the robotic arm to a different position without changing the position of the effector. The user input via the input and/or detection means is used in the controller so that the kinematic solution is optimized in real time and the robot follows the direction that the user places on the input and/or detection means. In particular, the input and/or detection means is fastened to a part of the robotic arm that would probably move in the case of a different solution from the amount of inverse kinematics solutions. In the present case, an adjustable robotic arm with end effector thus is provided, which ensures particularly good interaction.

Advantageous embodiments are explained in particular below.

According to one embodiment, the robotic arm may comprise at least two robotic arm segments/robotic arm members and the input and/or detection means may be provided at a joint or connection point, in particular at a joint and/or bearing (as connection), between the at least two robotic arm segments. In particular, the input and/or detection means may alternatively or additionally be arranged at a joint and/or bearing from robotic arm segment (of the robotic arm) to end effector. Alternatively or additionally, the input and/or detection means can be provided at a joint or connection point, in particular at a joint and/or bearing which connects the robot base to the robotic arm. Thus, while in at least two robotic arm segments the input and/or detection means can be arranged at a connection between these robotic arm segments, which do not form the joint respectively bearing to the robot base and do not form the joint respectively bearing to the end effector, alternatively or additionally input and/or detection means can also be provided at the first connection between the robot base and the robotic arm segment, or at the “last” connection between the robotic arm and the end effector. Due to the special arrangement of the interaction unit respectively the input and/or detection means at (one of) the connections respectively joints respectively bearings of individual members of the robotic arm, an interaction option can be provided at precisely those points that would most likely move as a kinematic solution of the robotic arm. These positions or points are therefore in particular the joints of the (robotic) arm, preferably at a joint between two robotic arm segments. If, for example, the input and/or detection means is arranged on a joint between two robotic arm segments, it moves with the robotic arm upon a movement in the area of the connection, while the end effector maintains its position, in particular its position and orientation (pose). In particular, there is an interaction unit/operating means in the form of the input and/or detection means for each point or joint.

According to a further embodiment, two input and/or detection means, in particular facing away from each other, can be provided on the robotic arm on at least one connection or connection point, whereby these input and/or detection means control a movement of the robotic arm in opposite directions or control a kinematic movement forwards and backwards. Preferably, there are even two input and/or detection means at each point or joint of the robotic arm, which act in opposite directions or perform opposite control of the robotic arm by the control unit. Opposite control can be, for example: “Move robotic arm left/right at the point (of the assigned input and/or detection means)”, “Rotate joint left/right at the point (of the assigned input and/or detection means)”. By providing two input and/or detection means intuitive and safe control thus can be provided, similar to a remote control with selection buttons at the top and bottom. In the case of opposing input and/or detection means, accidental incorrect control in one control direction can be excluded. In particular, the input and/or detection means thus can be provided or attached specifically to the opposite sides of a robotic arm, in particular an elbow of a 7DoF robot (robot with seven degrees of freedom), and the input respectively detection of an object of one of the means causes a self-movement in one direction, while the input respectively detection of an object of the other means causes a self-movement in the other, opposite direction.

In particular, a digital button/a digital pushbutton can be used respectively utilized as an input element. Among other things, by way of the digital button a sterile barrier at this point can be well integrated. In particular, the digital button is a button that detects a binary input signal (on and off) and forwards it to the control unit.

Preferably, a particularly pressure-sensitive button/pushbutton with an actuation direction and/or a pressure-sensitive control panel can be used/provided as input means. Input means thus can be used in particular that generate analog input signals, such as, for example, a pressure-sensitive surface, and that can also detect the amount of an input in addition to an input itself (on and off). In this way, the information of a strength for further processing can be gathered from an input signal. In particular, a linear relationship between input pressure and strength of the input signal (up to a limit value) can be provided. However, the control unit can also be adapted to process the input signal and, based on this, to interpose a stored reference curve in order to ensure adapted actuation. Moreover, the course of an input signal can be evaluated by the control unit and a control can be adapted based on the course of the input signal.

According to one embodiment, the control unit can be adapted to execute a movement, in particular a non-linear movement, of the robotic arm perpendicular to the direction of actuation when the button is actuated in the direction of actuation at the location of the input and/or detection means. The force exerted by a user on the button then does not result in a movement of the robot along the direction of the force, but in a different direction, including non-linear movements of the robotic arm. In this way, improved interaction can be provided to a user. For example, if a user leans forward and presses on an input means, the robotic arm with the input means does not move still further away from the user, which would make input more difficult, but instead moves approximately sideways or downwards. In this way, the input means remains within access of the user.

In particular, the button, which is pressure-sensitive, and/or the pressure-sensitive control panel can detect an amount of input pressure and the control unit can modulate a speed and/or a distance of a movement of connection points of the robotic arm on the basis of the detected pressure. In particular, an analog input signal hence can be used to modulate the speed of movement of the joints. The analog input signal can also be used to modulate a distance of movement of the joints. This means that the higher the pressure, the higher the speed respectively distance of the movement of the joints. There is therefore a proportional relationship in particular. A movement of the robotic arm can be controlled particularly precisely with only slight pressure, while the robotic arm can be moved quickly for rough orientation with high pressure.

According to a further embodiment, a distance sensor, in particular an ultrasonic sensor or a capacitive sensor, can be used as detection means, and the control unit can be adapted to move the robotic arm, in particular move it away from the object when an object is detected by the distance sensor, in particular when the distance is less than a predetermined limit value/threshold value. A capacitive sensor detects a change in electrical capacitance (such as in case of a touch display) and can thus detect an interaction (also contact-free). The limit value ensures that the robotic arm is not moved inadvertently, even though an object has only come close to the distance sensor unintentionally. In other words, one or more distance sensors, such as an ultrasonic sensor or a capacitive sensor, can be used in addition to or instead of input means, such as a pair of buttons. The distance sensor is utilized to create space for objects that come close to the distance sensor. In particular, the distance sensor is employed using a threshold value, so that the robot kinematics are only optimized when an object is below a certain distance.

Preferably, the control unit can modulate a speed and/or the distance of a movement of connection points of the robotic arm based on the detected distance. The detected distance between the sensor and the object hence is employed to modulate the speed of a movement of the joints or to modulate a distance between the robot joints. In this case, the modulation in particular is anti-proportional: the closer the object is to the distance sensor and the smaller the distance detected, the faster the speed of movement of the robotic arm is adjusted by the control unit.

According to a further embodiment, the at least one input and/or detection means, in particular a button, can be arranged coaxially to a joint axis, in particular a rotation axis, of the robotic arm. In this way, the input and/or detection means is arranged precisely on the joint and can control a movement at this point.

In particular, input and/or detection means, preferably buttons, may be specifically mounted to or on a linear axis or on the robot base of the robot if a linear axis is used to move the robot base. In this case, in particular, a pair of input and/or detection means can be used to move the robot base along the linear axis while maintaining the position, in particular the pose, of the end effector.

In particular, the input and/or detection means is an integral part of the robotic arm and is firmly connected thereto. If the input and/or detection means thus is built-in directly into the robotic arm an installation space can be further optimized and intuitive operation made easier.

With regard to a (computer-implemented) control method for controlling a medical collaborative robot having a robotic arm connected to a base and an end effector connected to the robotic arm, in particular according to the present disclosure, the task of the present disclosure is solved by the steps of: detecting an input or detecting an object by way of at least one input and/or detection means, in particular by way of a button and/or a distance sensor; and controlling and moving the robotic arm such that the end effector maintains its position, in particular pose, in space. The robotic arm is thus controlled in such a way that it moves out of an area to be kept clear on the basis of a stored kinematic algorithm, while the end effector maintains its position, in particular pose.

Preferably, the control method may comprise the steps of: detecting an operating pressure by means of a pressure-sensitive button and/or a pressure-sensitive control panel; and modulating a speed based on the detected input pressure/operating pressure. Analogous to the collaborative robot described above, the speed of a movement of the robotic arm can be adjusted by the input pressure. The higher the pressure, the faster the movement speed can be.

In particular, the control method can have the following step: detecting an input pressure by means of a pressure-sensitive button and/or a pressure-sensitive control panel; and modulating a distance between two points of the robotic arm, in particular between two joints and/or bearings of the robotic arm, based on the input pressure.

Alternatively or additionally, according to a further embodiment, the control method may comprise the step of: detecting a distance by means of a distance sensor; modulating a speed on the basis of the detected distance. In particular, the ratio is used that the smaller the distance, the higher the speed.

With regard to a computer-readable storage medium, the problem is thus solved in that it comprises instructions which, when executed by a computer, cause the computer to carry out the method steps of the control method according to the present disclosure. With respect to a computer program, the problem is solved by comprising instructions which, when executed by a computer, cause the computer to perform the method steps of the control method according to the present disclosure.

Any disclosure relating to the collaborative robot according to the present disclosure applies to the control method according to the present disclosure as well as vice versa.

### BRIEF DESCRIPTION OF THE DRAWINGS

The present disclosure will be described in more detail below with reference to the accompanying figures, with reference to preferred embodiments, wherein:

FIG. 1 is a perspective view of a medical collaborative robot according to a preferred embodiment;

FIG. 2 is a detailed perspective partial view of a swivel joint of the collaborative robot of FIG. 1;

FIG. 3 is a flowchart of a control method according to a preferred embodiment in a robot with an input means; and

FIG. 4 is a flowchart of a control method according to a further preferred embodiment in a robot with a detection means.

The figures are schematic in nature and only serve for comprehension of the invention. Identical elements are marked with identical reference signs. The features of the various embodiments can be interchanged.

### DETAILED DESCRIPTION

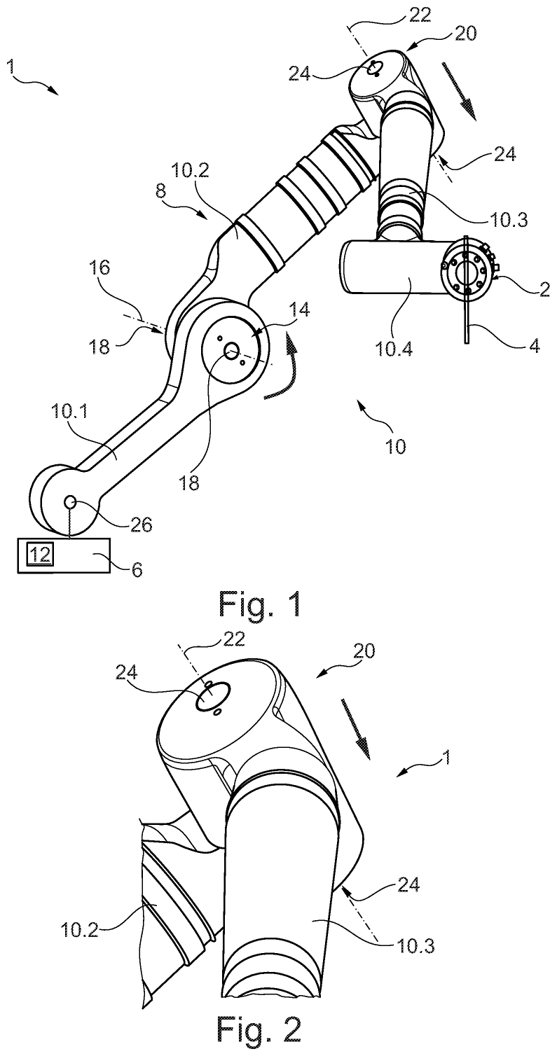

FIGS. 1 and 2 show a perspective view respectively a detailed perspective partial view of a medical collaborative robot 1 (hereinafter referred to as cobot) according to a preferred embodiment for actuating a medical end effector 2 that guides a surgical instrument 4.

The cobot 1 has a robot base 6 in the form of a stationary base arranged in space as the local connection point of the cobot 1. A multi-jointed/multi-link/multi-segmented robotic arm 8 is hinged to this robot base 6, to the end of which the end effector 2 with the instrument 4 is guidably hinged. In this embodiment, the robotic arm 8 with its robotic arm segments or robotic arm members 10 has more degrees of freedom in the axis space (DoF) than degrees of freedom to be manipulated, so that different poses of the robotic arm 8 are possible and adjustable starting from one pose of the end effector 2. In other words, the pose of the end effector 2 does not bijectively determine the pose of the robotic arm 8, but there is a solution space with a large number of different poses of the individual robotic arm segments 10 relative to each other and thus of the robotic arm 8. Thus, while the pose of the end effector 2 remains the same, the robotic arm 8 can be moved within the solution space in accordance with geometrically possible kinematics.

To control the cobot 1, the cobot has a control unit 12 that can control the robotic arm segments 10 of the robotic arm in order to move the robotic arm 8. Specifically, the robotic arm 8 has a first robotic arm segment 10.1, which is hinged to the robot base 6. A second robotic arm segment 10.2 is hinged to the first robotic arm segment 10.1 via a first swivel joint 14 with a first axis of rotation/joint axis 16.

In contrast to the prior art, on both sides an input means in the form of a pressure-sensitive (digital) button 18 (only one button 18 is shown, the other button is on the averted side of the first swivel joint 14) is respectively installed directly in the first swivel joint 14, aligned coaxially to the axis of rotation 16 and facing away from each other. As a result, two input means/operating elements are provided on respectively in the joint 14 itself on two different sides in the form of the two opposite buttons 18, which enable a surgeon to make a manual haptic input. Based on this input, the robotic arm segments 10 of the robotic arm 8 are then controlled via the control unit 12 in such a way that the position, in particular the pose of the end effector 2 remains unchanged despite the movement of the robotic arm 8.

Specifically, in this embodiment, manually pressing the button 18 on the swivel joint 14 would cause the first and second swivel joint segments 10.1 and 10.2, and thus the robotic arm 8, to rotate about its axis 16 (see arrow for direction of rotation) while maintaining the position of the end effector 2. To perform a self-movement in the opposite direction, the second button 18 on the other side of the robot 1 can be used.

Thus, the present concept provides for a specific mounting position of a pair of push buttons 18 on the first swivel joint 14. Pressing one of the buttons 18 causes the robotic arm 8 to rotate about its first axis of rotation 16, but in such a way that a position, in the present case even the pose (i.e. position and orientation) of the end effector 2 with the instrument 4 is maintained. Pressing the second button 18, which is coaxially opposite the first axis of rotation 16, causes an opposite movement to the first button 18. This provides the surgeon with a simple, safe and intuitive way of operating the robotic arm 8, for example, to move the robotic arm 8 away and improve accessibility to an intervention area.

The pressure-sensitive buttons 18 not only measure an operating input in binary form (input, no input), but also detect an applied pressure on the buttons 18. The amount of the applied manual pressure is further processed by the control unit 12 in such a way that it modulates, in this case correlates, a speed of movement of the robotic arm 8 with the pressure. Thus, if the button 18 is only pressed lightly, the robotic arm 8 moves slowly, whereas, if the button 18 is pressed strongly/firmly, the speed of movement (up to a maximum speed factor) is set high by the control unit 12. This allows the cobot 1 to be controlled even more precisely if required, but also faster if necessary.

A third robotic arm segment 10.3 is hinged to the second robotic arm segment 10.2 via a further, second swivel joint 20 with a second axis of rotation 22. On this second swivel joint 20, too, two distance sensors 24 facing away from each other on both sides are provided as detection means. Alternatively, instead of the distance sensors 24, buttons as on the first joint 14 can also be used. The distance sensors 24 again are arranged coaxially to the second axis of rotation 22 on both sides and are in the form of ultrasonic sensors. They are intended and adapted to detect a distance to an object, such as a hand, in their area and to make it available to the control unit 12 in a computer-readable manner.

Specifically, by means of the control unit 12, the cobot 1 is adapted to move the robotic arm 8 away from the object, in particular the hand, at the location of the second joint 20 when a distance of less than 5 cm is detected, or to perform an evasive movement, while the position, in particular the pose of the end effector 2 is not changed. This allows a user's hand to move closer to the first distance sensor 24 on one side of the joint 20 and the second and third robotic arm segments 10.2 and 10.3 move away from the hand. Different movement patterns or directions can also be set. Instead of a movement in a transverse direction along the second axis of rotation 22 a rotation about the axis of rotation 22 itself can also be controlled by the control unit 12, so that only a movement perpendicular to the axis of rotation 22 takes place.

In this embodiment, the control unit 12 is adapted to modulate the speed of movement of the robotic arm segments relative to each other based on the detected distance. The distance correlates anti-proportionally to the speed. If a movement is initiated by moving the hand towards the second swivel joint 20 with the distance sensor 24 at a distance of less than 5 cm, the movement speed of the robotic arm 8 can be set according to the distance. The closer the hand is to the distance sensor 24, the faster the robotic arm 8 moves at the location of the second swivel joint 20. The straight arrow in FIG. 1 shows a self-movement with the distance sensor at the elbow of the robotic arm 8. To perform a self-movement in the opposite direction, the opposite, second distance sensor 24 (not shown here, but indicated by reference sign 24) on the other, averted side of the robotic arm 8 is used again.

In particular, the control unit 12 can be adapted to maintain a constant distance. In this way, a modulated speed is set.

Finally, a fourth robotic arm segment 10.4 is provided on the third robotic arm segment 10.3, to which the end effector 2 with the instrument 4 is hinged.

Likewise, a button 26 is provided at the connection between the first robotic arm segment 10.1 and the robot base 6 in order to initiate a movement at this point. In one embodiment, the button 26 can also be used to perform a linear movement of the robot base 6 and thus of the connected robotic arm 8.

Thus, input means in the form of two averted buttons 18, 26 for two opposite directions of movement are respectively provided on the robotic arm 8 at each individual joint, starting from the robot base 6 via the individual robotic arm segments 10, and hence also detection means in the form of two averted distance sensors 24 and arranged directly in respectively on the joints 14, 20, approximately coaxially to these joints, so that the robotic arm 8 can be moved at any point of the joints 14, 20. The control unit 12 is adapted to control the individual robotic arm segments 10 of the robotic arm 8 via the control unit 12 during an input respectively detection of an object in such a way that the end effector 2 maintains its position and in particular its pose during the movement of the robotic arm 8.

In one embodiment, all buttons 18, 26 and distance sensors 24 can also be uniformly present as switchable input and detection means. More precisely, a hybrid of button and distance sensor can be present, which either allows manual direct operation by way of a button or, when switched over by the control unit, functions as a distance sensor and allows contactless actuation. In this way, it is possible to switch back and forth between operation by touch and operation without touch as required. If the surgeon is wearing sterile gloves during an operation and does not want to come into contact with the cobot 1, contactless operation can be set using the distance sensor, and if, for example, the robotic arm 8 is initially to be moved into the correct position, the touch-sensitive button can be selected.

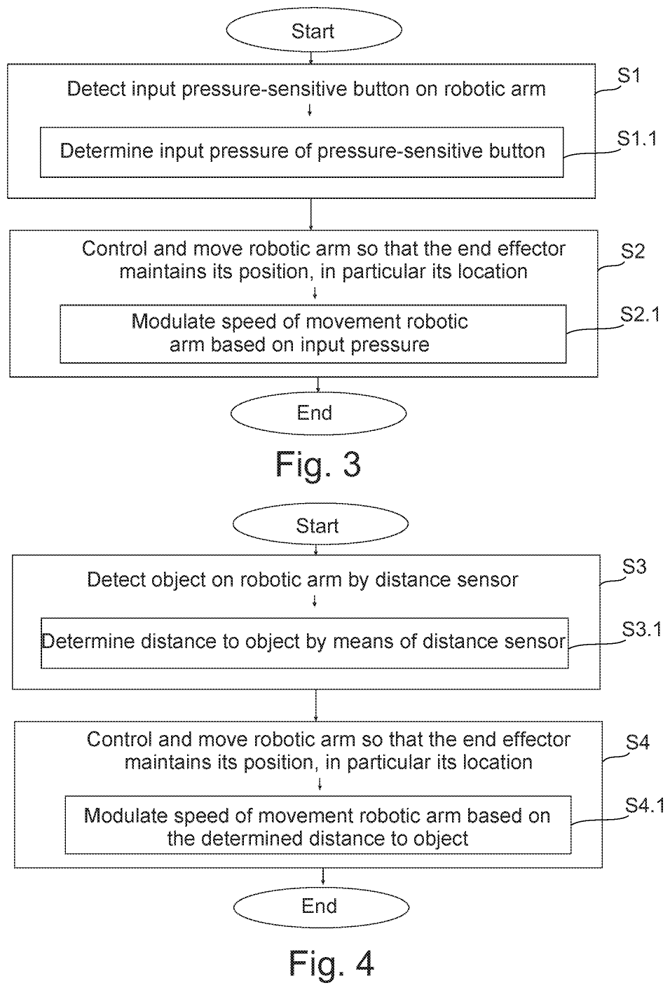

FIG. 3 shows a flowchart of a control method according to a first preferred embodiment, which can be used in particular with the cobot 1 of FIGS. 1 and 2. In the present case, a control method for a button is described.

In a first step S1, an input of a pressure-sensitive button 18, which is provided on a robotic arm is detected.

In a sub-step S1.1, in addition to the input itself, an input pressure/operating pressure of the button 18 is determined.

In a step S2, the robotic arm 8 is controlled and moved by the control unit 12 in such a way that the end effector 2 maintains its position, in particular its pose, in space.

In a sub-step S2.1, the speed of the movement of the robotic arm 8 is modulated on the basis of the detected operating pressure.

FIG. 4 shows a flowchart of a control method according to a further, second preferred embodiment, which can be used in particular with the cobot 1 of FIGS. 1 and 2. In the present case, a control method for a distance sensor is described.

In a first step S3, an object, such as a hand or an instrument, is detected by way of at least one detection means in the form of a distance sensor 24, which is provided on the robotic arm 8.

In a sub-step S3.1, the distance to the object is also detected by means of the distance sensor 24;

In a step S4, when an object is detected, the robotic arm 8 is again controlled and moved in such a way that the end effector 2 maintains its position, in particular pose, in space.

In a sub-step S4.1, the speed of the movement is modulated on the basis of the distance detected. If the distance decreases, the speed is increased; if the distance increases, the speed of movement of the robotic arm 8 is decreased until the limit value of the minimum distance to which movement no longer occurs.

It can also be said that with the above control method according to the second embodiment of the present disclosure, a contactless, manual remote control of the robotic arm 8 is possible, in which the robotic arm 8 is moved away from a direction of movement of the hand. Thus, a hand can be stretched in a direction along a path to an area of intervention and the path previously blocked by the robotic arm 8 is released by moving the robotic arm 8 out of the path.

1. A medical collaborative robot for actuating a medical end effector during a surgical procedure, the medical collaborative robot comprising:
a robot base as a local connection point of the medical collaborative robot;
a robotic arm that is movable and connected to the robot base;
an end effector which is connected to the robotic arm;
a control unit which is adapted to control and actuate at least the robotic arm; and
at least one input and/or detection means is arranged on a joint and/or bearing of the robotic arm as a connection,
the control unit, when an input respectively an object is detected, is adapted to control the robotic arm via the control unit in such a way that the end effector maintains its position during the movement of the robotic arm.

2. The medical collaborative robot according to claim 1, wherein:
the robotic arm has at least two robotic arm segments and the at least one input and/or detection means is provided on a joint and/or bearing as a connection between the two robotic arm segments, and/or
the at least one input and/or detection means is provided on a joint and/or bearing as a connection between the robot base and the robotic arm.

3. The medical collaborative robot according to claim 1, wherein two input and/or detection means facing away from each other are provided on the robotic arm on at least one connection, these controlling a movement of the robotic arm in opposite directions by means of the control unit.

4. The medical collaborative robot according to claim 1, wherein a button with an actuation direction and/or a pressure-sensitive control panel is used as input means.

5. The medical collaborative robot according to claim 4, wherein the control unit is adapted to perform, when the button is actuated in the actuation direction at the position of the button, a movement of the robotic arm perpendicular to the actuation direction in order to move the robotic arm away from this point.

6. The medical collaborative robot according to claim 4, wherein the button, which is pressure-sensitive, and/or the pressure-sensitive control panel detects an amount of an input pressure and the control unit modulates a speed and and/or a distance of a movement of connections of the robotic arm based on the detected input pressure.

7. The medical collaborative robot according to claim 1, wherein a distance sensor is used as detection means and the control unit is adapted to move the robotic arm away from an object when the object is detected by the distance sensor, when the distance is less than a predetermined limit value.

8. The medical collaborative robot according to claim 7, wherein the control unit modulates a speed and/or a distance of a movement of joints of the robotic arm based on the detected distance.

9. The medical collaborative robot according to claim 1, wherein the at least one input and/or detection means is arranged coaxially to a joint axis of the robotic arm.

10. A control method for controlling a medical collaborative robot having a robotic arm connected to a base and an end effector connected to the robotic arm, the control method comprising the steps of:
detecting an input or detection of an object by at least one input and/or detection means arranged on the robotic arm; and
controlling and moving the robotic arm in such a way that the end effector maintains its position in space.

11. The control method according to claim 10, further comprising the step of:
detecting an operating pressure by means of a pressure-sensitive button and/or a pressure-sensitive control panel as input means; and
modulating a speed of a movement of the robotic arm based on the detected operating pressure.

12. The control method according to claim 10, further comprising the steps of:
detecting a distance by means of a distance sensor as detection means; and
modulating a speed of a movement of the robotic arm based on the detected distance.

13. A computer-readable storage medium comprising instructions which, when executed by a computer, cause the computer to execute the steps of claim 10.

14. The medical collaborative robot according to claim 1, wherein the movable robotic arm has more degrees of freedom in the axis space than degrees of freedom to be manipulated.

15. The medical collaborative robot according to claim 1, wherein the end effector is connected to an end side of the robotic arm.

16. The medical collaborative robot according to claim 1, wherein the end effector maintains its pose during movement of the robotic arm.

17. The medical collaborative robot according to claim 7, wherein the distance sensor is an ultrasonic sensor or a capacitive sensor.
